(19) United States
(12) Patent Application Publication
(10) Pub. No.: US 2009/0248087 A1
(43) Pub. Date: Oct. 1, 2009

(54) Variable axis locking mechanism for use in orthopedic implants

(75) Inventors: Derek S. Lewis, Copley, OH (US); David B. Kay, Akron, OH (US)

Correspondence Address: HUDAK, SHUNK & FARINE CO. LPA, Suite 307, 2020 Front Street, Cuyahoga Falls, OH, 44221, US

(73) Assignee: OrthoHelix Surgical Designs, Inc., Akron, OH (US)

(21) Appl. No.: 12/380,339

(22) Filed: Feb. 26, 2009

Related U.S. Application Data
(60) Provisional application No. 61/067,952, filed on Mar. 3, 2008.

(51) Int. Cl. A61B 17/86

(52) U.S. Cl. 606/301

(57) ABSTRACT

A locking mechanism assembly to allow locking of a fastener in an orthopedic implant having its axis at a variable angle relative to the axis of a threaded through opening in the implant. The assembly includes a convexly rounded ring shaped locking insert which mates with the through opening. The head of the fastener includes a concentric flange that engages the locking ring into a locking relationship with the threads of the opening as the fastener is tightened into position. Preferably the locking ring is integral with the head of the fastener. The locking ring is made from a material that is more deformable than the material of the plate.

### CROSS-REFERENCE

This is a U.S. patent application of U.S. Provisional Application No. 61/067,952 filed on Mar. 3, 2008 for VARIABLE AXIS LOCKING MECHANISM FOR USE IN ORTHOPEDIC IMPLANTS which is hereby fully incorporated by reference.

### FIELD OF THE INVENTION

This present invention relates to a mechanism for allowing a screw or peg to be used in an orthopedic implant, such as a plate, at a variable axis and subsequently to be locked into a desired orientation into a threaded opening.

### BACKGROUND OF THE INVENTION

The field of orthopedics has included countless advances in the design of implants for internal fixation. The present invention provides an advance in the design of an assembly which allows a surgeon to choose whether to use a locking fixator including for example, a screw, or peg locked at a predetermined angle or to use a variable locking fixator inserted through a stabilizer, such as a plate, anchor or cage, at a variable angle in order to best capture a bone or bone segment with the fixator. The angle can subsequently be locked to fix the bone or bone segment relative to the plate, or to fix the plate relative to the bone or bone segment. The invention allows for at least about 25° and more preferably 30° of angulation relative to a longitudinal axis of the opening that the fixator is inserted through.

There are numerous applications which can benefit from such a mechanism. Specific examples include use in the small bones, such as the metacarpals and carpals, and the metatarsals and tarsals, although it is understood, that the mechanism can also be of great use in other areas of the body, including the long bones, the pelvis and the spine.

### SUMMARY OF THE INVENTION

The assembly of the present invention includes a convexly rounded, and optionally spherical locking ring that is seated in the concavely rounded corresponding opening in the implant. The locking insert is deformable relative to the opening in which it seats. In addition, it is preferable that the opening includes high friction deformation means, such as a series of internal threads that bite into the locking insert so as to deform the ring and bind it into position relative to the plate. This further provides the advantage that the locking insert can be used in a threaded locking opening, allowing the surgeon the option of using a threaded locking screw to lock the screw in the plate at a selected angle or to use the present assembly to select a different angle.

In particular, the fixator, may be a screw or peg, which includes a flange that extends radially outward and around the head of the screw or peg which compliments the profile of the locking ring and provides a surface that mates with a bevel in the opening of the locking ring. The head includes a slight taper on the exterior surface below the flange. The locking ring seats below the flange on the head and engages the tapered surface. The portion of the screw head which is axially proximal to the flange also compliments the locking ring so as to form a non-obtrusive or low profile in the screw hole. As the fixator is inserted into the plate, the flange presses the locking ring downward into a deformable contact with the internal threads in order to cause a friction fit of the locking insert in the opening and to lock the screw or peg at the desired angle. The ring and the screw are essentially one unit as delivered to the user. The ring is assembled onto the screw so as to remain integral to the screw head. Alternatively, the ring can be assembled into the opening, and the screw can be inserted through the ring to assemble the variable locking assembly. Preferred method of assembly is the use of a Morse taper (approx 6-7 degrees) on the screw head and the ring interior to fix the two components together. The ring and screw can be integrated together by mechanical means including the interference fit previously mentioned, by other interlocking means including for example, locking flanges or friction surfaces, or chemical bonding can be used, including adhesives. Additionally, the ring will preferably have a geometry that fits onto the screw head to prevent rotation of the ring with respect to the screw during insertion. The ring could be either machined and assembled or over-molded onto the screw.

### BRIEF DESCRIPTION OF THE DRAWINGS

FIG. 1 is a cross section of a plate illustrating embodiment of the variable axis locking mechanism assembly in accordance with the present invention;

FIG. 2 is a perspective view from the top and the side of a locking insert of the present invention;

FIG. 3 is a side view of the locking insert of FIG. 2; and

FIG. 4 is a side view of a screw that can be used as part of the locking mechanism assembly of the present invention.

### DETAILED DESCRIPTION OF THE INVENTION

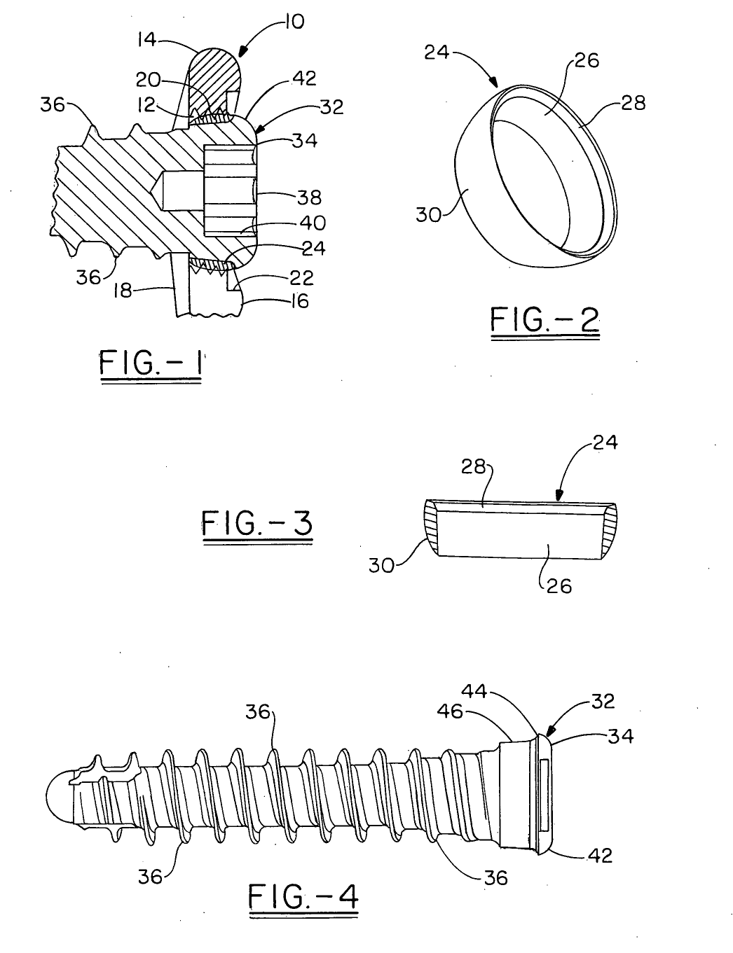

FIG. 1 shows a variable axis locking mechanism assembly 10 in an opening 12 in the plate 14 which is representational of any number of plate designs that can use the present invention. The plate has a top surface 16 and a bottom surface 18 (which is the surface that opposes, if not engages the bone or bone segments for which the plate is used.) The plate includes an opening 12 that extends from the top surface 16 though to the bottom surface 18 which includes internal threads 20 and a counterbore or recess 22 which provides a seat for the use of a drill guide and which provides a zero point in the inspection of the taper depth during manufacture. While the opening is threaded, it is shaped for a threaded member having a taper in both the major and the minor diameter.

The locking ring 24 has an internal opening 26 with a bevel 28 that leads into the opening. The external surface 30 of the locking ring is rounded and together with a rounded shoulder of the fixator 32 forms a smoothly rounded profile to accommodate a variable angle and which has no sharply projecting edges to would irritate soft tissue after implantation. The fixator 32 is in this case a screw having a head 34 and distal threads 36. The head includes a top surface 38 with an internal torque driving recess 40. The top surface 38 includes a rounded shoulder 42 which is under cut to form a concentric flange 44. The flange joins a side wall 46 having a circular cross section, but which tapers toward the distal end of the screw. The taper is from about 5° to about 15°, and preferably about 6° to about 10°. The screw includes a necked area which joins the head to a threaded are which includes a cancellous bone screw 48. The screw head can also include other means which will couple the locking insert and the screw head, including for example, a series of concentric flanges, knurling, or threads.

The locking ring shaped insert, having smoothly rounded exterior walls 30, which optionally can include a higher friction surface as is created by knurling, milling or otherwise roughening or texturing the surface. The locking ring is made from a material, which is relatively deformable compared to the plate in which it seats. Thus, the insert will deform rather than splinter as it engages the seat. For example, the locking ring may be made of a biocompatible polymer or ceramic, such as as PEEK or UHMWPE (ultra high molecular weight polyethylene), or a softer metal, such as stainless or commercially pure titanium in a titanium alloy plate.

While in accordance with the patent statutes the best mode and preferred embodiment have been set forth, the scope of the invention is not limited thereto, but rather by the scope of the attached claims.

What is claimed is:

1. A variable angle locking mechanism assembly for use in an orthopedic implant, comprising:
the implant made of a first material and having a through opening having a longitudinal axis, the opening including internal threads;
a ring shaped insert made of a second material and having a central opening and having a convexly rounded side wall and sized to fit in the through opening in the implant at a plurality of angles relative to the longitudinal axis of the through opening; and
a fastener that is adapted to extend through and engage the central opening of the insert and having a head that includes an undercut to form a circular or helical flange member which mates with the locking insert to selectively lock the insert at an angle in the through opening relative to the longitudinal axis as the side wall of the insert engages the internal threads of the through opening.

2. The variable angle locking mechanism assembly as set forth in claim 1, wherein the second material is more deformable than the first material.

3. The variable angle locking mechanism assembly as set forth in claim 1, wherein the fastener is a peg or a screw.

4. The variable angle locking mechanism assembly as set forth in claim 3, wherein the locking insert includes a proximal bevel and the head of the fastener includes a concentric flange that engages the bevel of the locking insert and further where the proximal side of the flange is rounded in the proximal direction such that the locking insert compliments the head of the fastener.

5. The variable angle locking mechanism assembly as set forth in claim 1, wherein the locking insert is integral to the fastener.

6. The variable angle locking mechanism assembly as set forth in claim 5, wherein the locking insert has an interference fit with the fastener.

7. The variable angle locking mechanism assembly as set forth in claim 6, wherein the fastener is a peg or a screw which has a head that has an exterior surface having a taper.

8. The variable angle locking mechanism assembly as set forth in claim 1, wherein the locking ring insert has an interior surface that has a taper.

9. The variable angle locking mechanism assembly as set forth in claim 1, wherein the locking ring insert is bonded to the fastener.

10. The variable angle locking assembly as set forth in claim 9, wherein the first material is metal and the second material is not metal.

11. The variable angle locking assembly as set forth in claim 10, wherein the second material is a ceramic or a polymer.

12. The variable angle locking assembly as set forth in claim 9, wherein the first material is an alloy of titanium and the second material commercially pure titanium.

13. A method of making a variable angle locking mechanism assembly for use in an orthopedic implant, comprising the steps of:
making an the implant made of a first material and having a through opening having a longitudinal axis and a concavely rounded wall about the longitudinal axis, the opening including internal threads;
making a ring shaped insert made of a second material which is more deformable than the first material and having a central opening and having a convexly rounded side wall and sized to fit in the through opening in the implant at a plurality of angles relative to the longitudinal axis of the through opening;
making a fastener that is adapted to extend through and engage the central opening of the insert and having a flange member which mates with the locking insert to selectively lock the insert at an angle in the through opening relative to the longitudinal axis as the side wall of the insert engages the internal through opening; and
assembling the ring shaped insert on the fastener to form an integral unit between the ring shaped insert and the fastener or assembling the insert within the implant to form an integral unit between the ring shaped insert and the implant.

14. The method of making the variable angle locking mechanism assembly as set forth in claim 13, wherein the locking insert has an interference fit with the fastener.

15. The method of making the variable angle locking mechanism assembly as set forth in claim 14, wherein the fastener is a peg or a screw which has a head that has an exterior surface having a taper or wherein the locking ring insert has an interior surface that has a taper.

16. The method of making the variable angle locking mechanism assembly as set forth in claim 14, wherein the flange member is a thread.

17. The method of making the variable angle locking mechanism assembly as set forth in claim 13, wherein the locking ring insert is bonded to the fastener.

18. The method of making the variable angle locking mechanism assembly as set forth in claim 17, wherein the locking ring insert is made on the fastener.

19. The method of making the variable angle locking assembly as set forth in claim 18, wherein the first material is metal and the second material is not metal.

20. The method of making the variable angle locking assembly as set forth in claim 19, wherein the second material is a ceramic or a polymer.

21. The method of making the variable angle locking mechanism assembly as set forth in claim 20, wherein the locking ring insert is made on the fastener.

22. The method of making the variable angle locking assembly as set forth in claim 21, wherein the first material is an alloy of titanium and the second material commercially pure titanium.
